(19) United States
(12) Patent Application Publication
(10) Pub. No.: US 2025/0292019 A1
(43) Pub. Date: Sep. 18, 2025

(54) System and Method for Flowsheet Population

(71) Applicant: Microsoft Technology Licensing, LLC, Redmond, WA (US)

(72) Inventors: Nathan Bodenstab, Eliot, ME (US); Eric Marshall, Bellevue, WA (US); Arlie E. Coles, Rowlett, TX (US); John Anderson, Burlington, MA (US); William Ham, Pittsburgh, PA (US); Seppo Andrew Rapo, Barnstable, MA (US); Georgios Michalopoulos, Waterloo (CA); Charles Grant Curtis, III, Seattle, WA (US)

(21) Appl. No.: 18/989,636

(22) Filed: Dec. 20, 2024

Related U.S. Application Data
(60) Provisional application No. 63/564,356, filed on Mar. 12, 2024.

(51) Int. Cl. G06F 40/279; G06F 16/21; G16H 10/60

(52) U.S. Cl. CPC G06F 40/279; G06F 16/211; G16H 10/60

(57) ABSTRACT

A method, computer program product, and computing system for flowsheet population. A structured document conforming to a schema and having a plurality of rows, each row comprising a key and a value, is processed. A target key/value pair is identified within the structured document for a target instance of information. An in-context example of spoken text representative of the target instance of information corresponding to the target key/value pair is generated using a generative artificial intelligence (AI) model.

### RELATED CASES

This application claims the benefit of U.S. Provisional Application No. 63/564,356 filed on Mar. 12, 2024, the contents of which are all incorporated by reference.

### BACKGROUND

In the context of documentation application for patient care, nurses must complete a flowsheet in the electronic health record (EHR) to document and track a patient's clinical data over time. This flowsheet contains many rows that can be filled with different kinds and types of information about the patient (e.g. blood pressure, temperature, etc.). All rows that can be filled with information are defined in a schema that is specific to each hospital. This hospital-specific schema can contain thousands of rows, but in a specific context only a small number of rows are relevant. Manually determining which rows are relevant and inserting appropriate data in those rows is reduces the efficacy of nursing personnel.

### BRIEF DESCRIPTION OF THE DRAWINGS

FIG. 1 is a diagrammatic view of a flowsheet population system;

FIG. 2 is a depicts a sample structured document (e.g., a flowsheet);

FIG. 3 is a flowchart of an embodiment of the flowsheet population process;

FIG. 4 depicts forms information in a transcript and in an associated structured flowsheet;

FIG. 5 is a flowchart of an embodiment of the flowsheet population process;

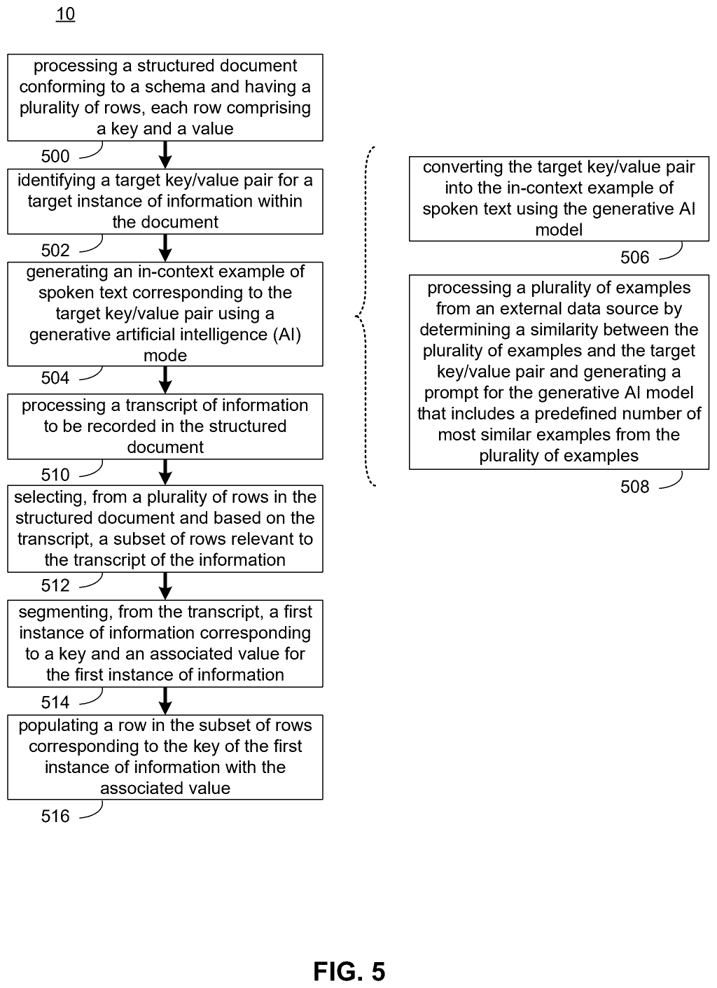

FIG. 6 depicts forms information in a transcript and in an associated structured flowsheet with an in-context example; and

FIG. 7 is a diagrammatic view of a computer system and the operating system and flowsheet population process coupled to a distributed computing network.

Like reference symbols in the various drawings indicate like elements.

### DETAILED DESCRIPTION

Documentation in many fields is defined by a structured document or flowsheet. In the example field of healthcare, medical or clinical documentation is tracked in an electronic medical record (EHR), where a user or healthcare professional updates the education provided, updates the care team with progress notes regarding the patient's condition, documents significant events, and details medical assessments in structured documents.

A flowsheet is a structured document used in various fields, including healthcare, engineering, and manufacturing, to record and track the progression of processes, procedures, and/or events over time. In healthcare, a flowsheet is commonly used to document and monitor patient data, particularly vital signs, measurements, and clinical assessments, in a systematic and organized manner. It typically consists of a grid or table format with rows representing different parameters or variables being measured and columns representing different time intervals, such as hours, days, or work shifts.

Each flowsheet can have multiple tabs and hundreds or even thousands of rows, with each row representing a specific data point. Working with flowsheets is a time-consuming process, since the user must first navigate to the appropriate tab in the flowsheet, identify the row or group of rows corresponding to the assessment that is being documented, then enter the information cell-by-cell. Health care professionals often do not document in flowsheets while delivering care and instead postpone this task, which can increase cognitive burden, extend the time it takes to chart, delay when this information is accessible to other clinicians, and increase the potential for inaccuracies. Within a healthcare setting, flowsheets may include categories for recording vital signs such as blood pressure, heart rate, temperature, respiratory rate, oxygen saturation, etc. Additionally, flowsheets may incorporate clinical assessments, interventions, medications administered, fluid intake and output, and other relevant patient information.

### Flowsheet Population

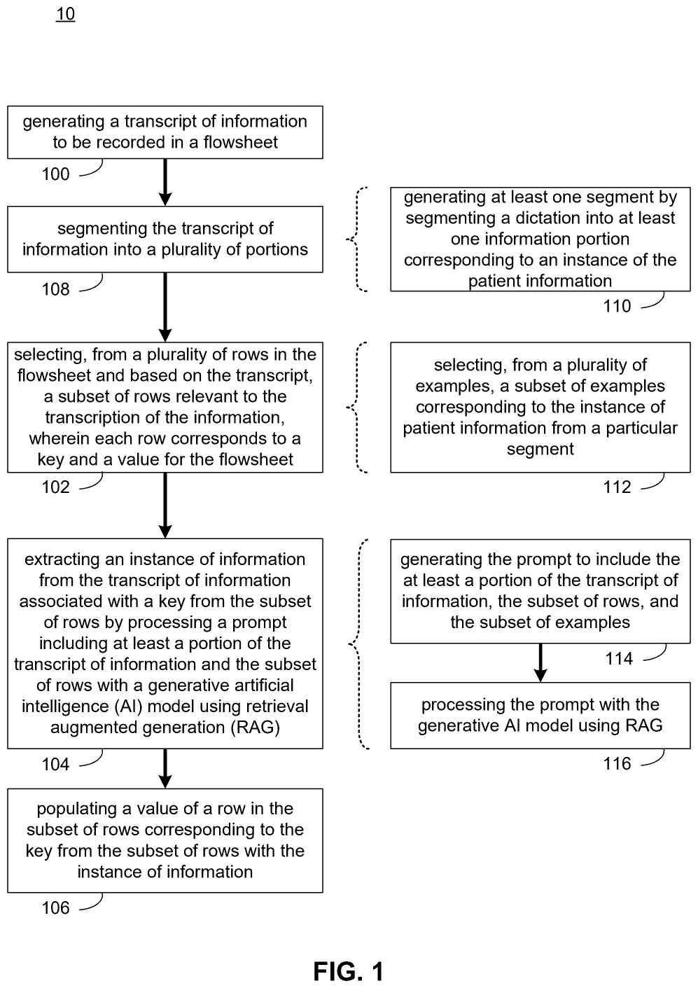

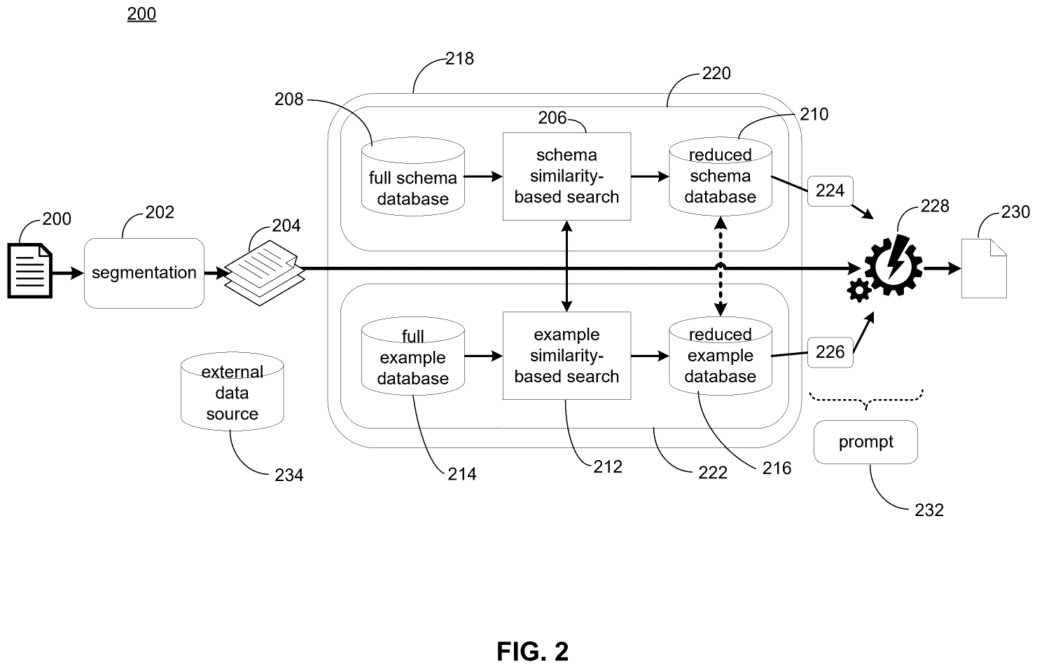

Referring to FIG. 1, a flowchart describing an implementation of flowsheet population process 10 is shown. In some implementations, flowsheet population process 10 generates 100 a transcript of information that includes instances of information that are to be inserted into a flowsheet, such as flowsheet 200 of FIG. 2. Flowsheet population process 10 selects 102, from a plurality of rows in the flowsheet and based on the transcript of information, a subset of rows relevant to the transcript of the information, wherein each row corresponds to a key and a value for the flowsheet. An instance of information is extracted 104 from the transcript of information associated with a key from the subset of rows by processing a prompt including at least a portion of the transcript of information and the subset of rows with a generative artificial intelligence (AI) model using retrieval augmented generation (RAG). A value of a row in the subset of rows corresponding to the key from the subset of rows is populated 106 with the instance of information.

Referring again to FIG. 1, flowsheet population process 10 generates 100 a transcript of information (e.g., transcript 200) through processing, transcription, and diarization of speech signals from one or more users. In some implementations, an optimized Speech-to-Text artificial intelligence (AI) model converts natural input speech from one, or multiple speakers, to textual form. In one example, the transcript is generated using an automated speech recognition (ASR) system from a dictation from a healthcare provider. In another example, the transcript is generated using an ASR system from a conversation between at least one healthcare provider and a patient. The input speech sequence may contain both meaningful and non-meaningful parts for flowsheet documentation. Meaningful speech includes verbalizations which are directly relevant to the flowsheet fields for a given patient, while non-meaningful speech includes any other irrelevant speech to the flowsheet documentation. The information extraction step that follows speech-to-text, can extract the meaningful information for the flowsheet documentation and reject the irrelevant information.

The audio capture device can be a mobile phone, in-room microphone array, desktop microphone, wearable device, or any other audio capture device. In some implementations, a recording device is positioned adjacent to the two speakers to capture the audio from both parties, an omnidirectional microphone can capture all audio in the vicinity, and an artificial intelligence (AI) model can diarize the speech by both speakers and ASR can transcribe all speech.

A microphone array may be used, in which the room can be equipped with wall mount microphone array. Alternatively, a portable microphone array built-in to a computing device is used. In one example, the microphone array is used to track non-stationary speakers by beamforming technology. In such a scenario, target speakers are pre-enrolled by voice biometrics. The diarization process tags target speakers, whose voice biometrics match, and other all non-enrolled speakers' speech are marked as unrecognized speakers. In one example, the ASR system transcribes only targeted speakers' speech.

In some implementation, flowsheet population process 10 segments 108 a first instance of information corresponding to a key and an associate value. For example and as discussed above, flowsheet population process 10 segments 108 the streaming or recorded transcript into contextually coherent, continuous and possibly overlapping segments such as phenomes, words, or phrases, via an artificial intelligence (AI) model. In one example, a prompted large language model (LLM) is instructed to divide the input text into contiguous segments, with the goal of keeping all related information together in one segment. In some implementations, segmenting 108 includes identifying pauses, changes in pitch, or other acoustic features that indicate the boundaries between different linguistic units. Effective segmentation improves the accuracy of speech recognition by ensuring that the system can better handle variations in speech, such as different speaking speeds, accents, and sentence structures. It also aids in improving the alignment between spoken words and their corresponding text, facilitating more precise transcriptions in ASR systems.

In some implementations, segmenting 108 the first instance of information includes generating 110 at least one segment by segmenting the dictation into at least one information portion corresponding to an instance of the patient information. As discussed above and in the example where the flowsheet concerns patient information for a healthcare provider, flowsheet population process 10 processes a dictation prepared by or for a healthcare professional concerning a patient. In this example, flowsheet population process 10 generates 110 segments the dictation into information portions or segments corresponding to particular instances of patient information. Referring also to FIG. 2 and in some implementations, the transcript is broken down into segments by a segmentation process 202, which decomposes the transcript into smaller substrings prior to processing.

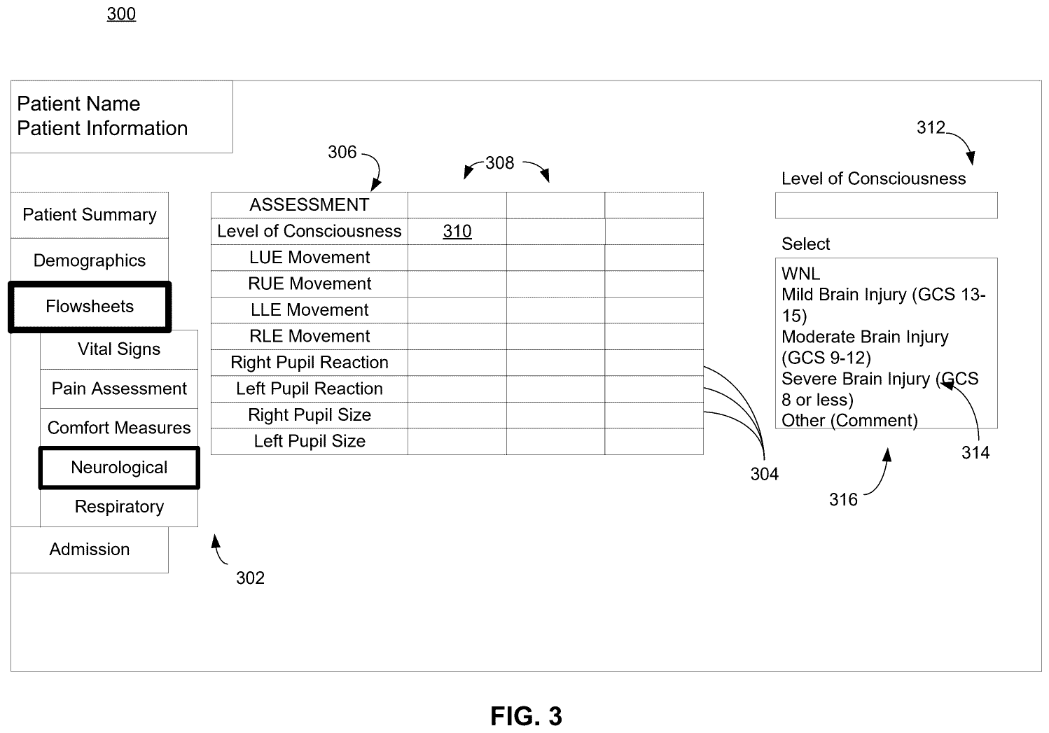

In some implementations, flowsheet population process 10 selects 102, from a plurality of rows in the flowsheet and based on the transcript, a subset of rows relevant to the transcript of the information. Referring also to FIG. 3, an example flowsheet 300 according to an embodiment of the disclosure is shown. Flowsheet 300 includes a listing of groups of data 302 associated with various information that will be contained in the flowsheet. Within each group, a number of rows 304 include key/value information for specific instances of information, designated at column 306. Within each column 308, information for different instances during an observation period are input for each row. Field 310 shows an example field into which a value would be input corresponding to the key “Level of consciousness.” Information may be input into field 310 (or any other field in the flowsheet) in the form of an integer, a float figure, string, date, time, selection from a single-choice list, a selection from a multi-choice list, or any other custom data type. In flowsheet 300, section 312 indicates a field in which a variable or freeform input may be entered. Window 314 is a multi-choice list, from which a user may select the input, and field 316 allows for the entry of comments in addition to the value input. In some implementations, flowsheet population process 10 uses a schema similarity-based search (e.g., schema similarity-based search section 206), to identify the most relevant rows in a full schema database (e.g., full schema database 208) and stores them in the reduced schema database 210 for use in connection with matching information in each segment with the appropriate row (key/value pair) in the flowsheet.

In some implementations, selecting 102 the subset of rows includes selecting 112, from a plurality of examples, a subset of examples corresponding to the instance of patient information from a particular segment. For instance and as shown in FIG. 2, flowsheet population process 10 performs example similarity-based search (e.g., example similarity-based search section 212) using a database (e.g., database 214) of examples for storing the full set of examples. In one example, flowsheet population process 10 processes segments of an input transcript to identify the most relevant examples in the full example database 214 and stores them in the reduced example database 216 for use in connection with matching information in each segment with the appropriate row (key/value pair) in the flowsheet corresponding to instances of patient information.

For example and referring again to FIG. 2, flowsheet population process 10 selects 102 the subset of rows using a retrieval augmented generation (RAG) portion 218, including a schema row selection section 220 and an in-context example selection section 222. Schema row selection section 220 includes a database 208 for storing the full schema of the hospital flowsheet, which can include thousands of rows of the flowsheet. A schema similarity-based search section 206 processes segments of an input transcript to identify most relevant rows in the full schema database 208 and stores them in the reduced schema database 210 for use in connection with matching information in each segment with the appropriate row (key/value pair) in the flowsheet. Example selection section 222 includes a database 214 for storing the full set of examples. An example similarity-based search section 212 processes segments of an input transcript to identify the most relevant examples in the full example database 214 and stores them in the reduced example database 216 for use in connection with matching information in each segment with the appropriate row (key/value pair) in the flowsheet.

In some implementations, the transcript segments 204 are input to schema similarity-based search section 206 and example similarity-based search section 212 for use in determining into which rows 224 the instances of information will be inserted and to determine which examples 226 would be beneficial in making the row-similarity decisions. Similarity-based search is used to retrieve items or data in each database 208, 214 that are similar to each input segment, based on specific features or characteristics of the segment. Rather than searching for exact matches, this approach uses algorithms to measure the degree of similarity between the input segment and the items in the databases 208, 214. The goal is to find rows and examples from databases 208 and 214, respectively, that share certain patterns, structures, or features with the input segment. For instance, in image search, a similarity-based search might compare visual features like color, shape, or texture to find images that resemble a target image. This type of search is valuable when exact matches are rare or insufficient, allowing for more flexible and intelligent retrieval of relevant results.

In some implementations and in the example of FIG. 2, rows 224 represent a subset of the rows in the full schema database 208. Based on the similarity-based search 206, the rows in the flowsheet having key/value pairs most similar to the segment being processed are selected and stored in reduced schema database 210. Likewise, based on the similarity-based search 212, the examples in full example database 214 that are most similar to the segment being processed are selected and stored in reduced examples database 216.

In some implementations, flowsheet population process 10 extracts 104 an instance of information from the transcript of information associated with a key from the subset of rows by processing a prompt including at least a portion of the transcript of information and the subset of rows with a generative artificial intelligence (AI) model using retrieval augmented generation (RAG). RAG combines the strengths of retrieval-based methods and generative models to enhance the quality and relevance of generated text. In RAG, a generative AI model, typically a large language model, is augmented with a retrieval component, allowing it to retrieve and incorporate relevant information from a large external knowledge source, such as full schema database 208, during the generation process. The retrieval component retrieves relevant context based on the input prompt or query, which is then used to guide the generation of text by the generative model. By leveraging external knowledge, RAG models can produce more informative, coherent, and contextually relevant responses compared to traditional generative models. Moreover, the retrieval mechanism enables RAG models to handle open-domain conversational tasks more effectively, as they can access a broader range of information beyond what is explicitly encoded in their parameters.

In some implementations, determined rows 224, determined examples 226, and segments 204 are input into a prompt of a large language model (e.g., generative AI model 228), which performs an extraction process to extract data from the segments and, as will be discussed in greater detail below, generate the flowsheet with the instances of information from each segment populated into appropriate rows of the flowsheet. In one example, the flowsheet is a structured JSON document 230.

To reduce latency and cost, and to stay within downstream context window constraints, the size of the schema to extract into is minimized by filtering potential rows to the top, for example, “N” candidates given the current segment with a retrieval-augmented generation (RAG) filtering method 220. The top “N” candidate rows are generated by measuring the similarity (e.g., cosine similarity, Euclidean distance, dot product similarity, etc.) between the segment 204 and each schema row, projecting each into generative AI model 228's embedding space. For example, the “N” most similar rows identified in schema similarity-based search process 110 are used to create a just-in-time prompt (e.g., prompt 234) for later data extraction, containing only relevant schema components.

In some implementations, extracting 104 the instance of information includes: generating 114 the prompt to include the at least a portion of the transcript of information, the subset of rows, and the subset of examples; and processing 116 the prompt with the generative AI model using RAG. For example, prompt 234 is generated to include in-context examples 226. An in-context example refers to an input-output pair (e.g. an input spoken text segment and the corresponding output structured data). These examples are useful for improving the robustness and generalization capabilities of structured data extraction, where variability in speaking patterns may change from user to user or over time.

In some implementations, when a new hospital is being adapted for processing according to embodiments of the disclosure, the hospital's schema is provided but no supervised examples may exist to retrieve for in-context learning. In this situation, a generative AI model (e.g., generative AI model 228) is leveraged to generate synthetic examples for each row in the hospital schema with the help of an existing example database for a sample schema. In some implementations, this is done in two steps. First, for each row in the hospital schema, a row embedding is generated, then its most similar rows from the sample schema are selected by the similarity (e.g., cosine similarity, Euclidean distance, dot product similarity, etc.) of their embeddings. Second, generative AI model 228 is prompted with examples containing those rows from the sample schema, using, for example, chain-of-thought prompting to force the model to generate a mapping, step-by-step, by first identifying the specific attributes of the row (e.g. datatype, value) that it must use before generating an appropriate transcript to be paired with the corresponding similar hospital schema row.

A generative AI model 228 (e.g., a prompted large language model (LLM)) is used to extract 104 the relevant structured text from the transcript segment 204, rows 224, and examples 226, and canonicalize it based on the schema. The extraction process isolates the needed information while discarding irrelevant data. Canonicalization follows extraction and involves standardizing the data into a consistent and uniform format, regardless of its original structure. In one example, this includes normalizing variations in data representation, such as converting all dates into a single format, standardizing case sensitivity, or resolving different identifiers for the same entity (e.g., “NYC” and “New York City”). Canonicalization ensures that all extracted data conforms to the flowsheet schema, despite how it was spoken. For example, the segment “the patient has a risk of fracture in his left arm” is converted to {“BoneFractureRiskOrInjury”: “1=Yes”} as the schema specifies two pick-list items for this row as the strings “0=No” and “1=Yes”.

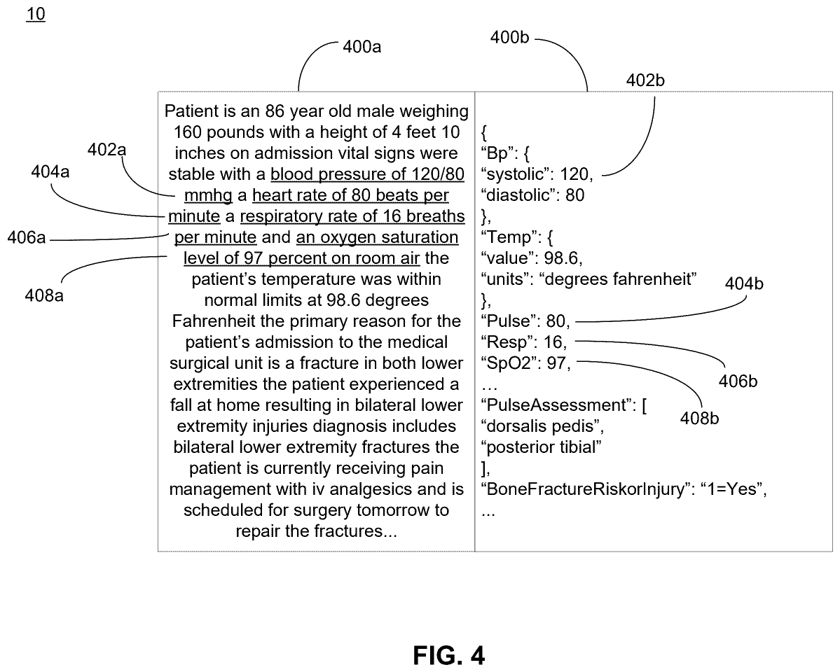

In some implementations, flowsheet population process 10 populates 106 a row in the subset of rows corresponding to the key of the first instance of information with the associated value. For example, FIG. 4 depicts an example of flowsheet population process 10, in which instances of information in a transcript are populated into rows (key/value) of a structured document (e.g., flowsheet). A portion 400a of a transcript is shown, along with the corresponding portion 400b of the structured flowsheet. Indicated at 402a is a segment of the transcript 400a indicating a “blood pressure of 120/80 mmhg.” After processing of the transcript 400a as described above, the information in this segment 402a is inserted into the key and value fields in the associated row 402b of the flowsheet 400b. In the associated row 402b in the flowsheet 400b, the key “Bp” (for blood pressure) is inserted and the values 120 (systolic) and 80 (diastolic) are entered. Likewise, the instance of information in segment 404a is inserted into the key and value fields in the associated row 404b of the flowsheet 400b. In the associated row 404b in the flowsheet 400b, the key “Pulse” and the value “80” are entered. The instance of information in segment 406a is inserted into the key and value fields in the associated row 406b of the flowsheet 400b. In the associated row 406b in the flowsheet 400b, the key “Resp” and the value “16” are entered. In this example, the instance of information in segment 408a is inserted into the key and value fields in the associated row 408b of the flowsheet 400b. In the associated row 408b in the flowsheet 400b, the key “SpO2” and the value “97” are entered. In this manner, flowsheet population process 10 automatically populates rows of the flowsheet by mapping segments of a transcript to key/value portions of the schema of the flowsheet.

While embodiments of the disclosure have been described mainly with regard to the application to hospital flowsheets for use by medical personnel, the system and method described herein may be utilized in connection with any environment in which freeform speech or unstructured text information is to be entered into a structured document conforming to a schema.

### Generating In-Context Examples From Complex Schema

In some implementations, flowsheet population process 10 processes 500 a structured document conforming to a schema and having a plurality of rows, each row comprising a key and a value. A target key/value pair is identified 502 within the structured document for a target instance of information. An in-context example of spoken text representative of the target instance of information corresponding to the target key/value pair is generated 504 using a generative artificial intelligence (AI) model. For example, an implementation of flowsheet population process is directed to generating in-context examples from exterior sources to assist in the identification of keys relevant to the information in each segment of a transcript. Retrieval augmented generation (RAG) is most effective when the document and the query text are in the same domain. For information extraction from a structured schema (e.g., a flowsheet), the input text is typically in natural language, while the schema (i.e., the documents to be retrieved) is in a structured format. As discussed above, examples from a full example database (e.g., full example database 214) are able to bridge spoken text with corresponding schema entries. However, there are many cases where this database does not exist or lacks sufficient example entries to provide effective examples for RAG as discussed above. Accordingly, implementations of flowsheet population process 10 use a generative AI model to convert structured schema data to possible spoken language.

In some implementations, flowsheet population process 10 processes 500 a structured document conforming to a schema and having a plurality of rows, each row comprising a key and a value. For example and as described above, a structured document conforms to a schema with various rows. In one example, the structured document is a flowsheet with rows that correspond to patient information. With each row of the schema, there is a key and a value. The key is a unique identifier for a record in a database and the value is the actual data stored in the record associated with the key.

In some implementations, flowsheet population process 10 identifies 502 a target key/value pair within the structured document for a target instance of information. For example and as shown in FIG. 1, flowsheet population process 10 uses the combination of key/value pairs from full schema database 208 and corresponding examples from full example database 214 to map instances of information from segments of a transcript to key/value pairs of the structured document. In some implementations, certain key/value pairs may lack any corresponding examples or a threshold number of examples. Accordingly, flowsheet population process 10 maintains a link between key/value pairs and examples within full example database 214. Flowsheet population process 10 identifies 502 a target key/value pair within the structured document by processing a number of examples within full example database 214 associated with each key/value pair. In response to determining that the number of examples is below a predefined threshold (e.g., zero examples, one example, etc.), flowsheet population process 10 identifies a key/value pair as a target key/value pair for generating an in-context example for a target instance of information.

Consider the following example where a group within the structured document concerns neurological patient information with a key “R Pupil Reaction” and a value “Reactive”, “Brisk”. In this example, a corresponding instance of information that might be spoken by a healthcare professional would include “Right Pupil Reaction is reactive, brisk”. However, a match between the spoken text and key/value pairs may not be straight-forward. For example, consider the example where a group within the structured document concerns safety with a key “Safety Promotion” and a value “bed alarm”. In this example, a healthcare provider may say “bed checked”. However, one must have domain knowledge to know that “bed checked” indicates that the bed alarm was checked as part of a safety review.

In some implementations, flowsheet population process 10 generates 504 an in-context example of spoken text representative of the target instance of information corresponding to the target key/value pair using a generative artificial intelligence (AI) model. For example, flowsheet population process 10 provides the target key/value pair to generative AI model 228 for generating an in-context example of spoken text representative of the target instance of information corresponding to the target key/value pair. In one example, flowsheet population process 10 generates a prompt with the target key/value pair and query to generate an in-context example of spoken text. An in-context example of spoken text is what an ASR system would likely generate as an instance of information for a particular key/value pair.

In some implementations, generating 504 the in-context example includes converting 506 the target key/value pair into the in-context example of spoken text using the generative AI model. For example, generative AI model 228 processes the target key/value pair by converting 506 the target key/value pair into an in-context example of spoken text. In one example, flowsheet population process 10 converts 506 the key “R Pupil Reaction” and value “Reactive”, “Brisk” to an in-context example by using the value to describe the key (i.e., “Right Pupil Reaction is reactive, brisk”).

In some implementations, generating 504 the in-context example includes processing 508 a plurality of examples from an external data source by determining a similarity between the plurality of examples and the target key/value pair and generating a prompt for the generative AI model that includes a predefined number of most similar examples from the plurality of examples. In one example, the in-context example is generated from an external data source (e.g., external data source 234) that is external from the structured document conforming to the structured schema. Examples of external data source 234 include documentation from the same domain as the structured schema (e.g., an external medical record in the medical domain for a flowsheet of a hospital; an external accounting report in a financial domain for a structured schema concerning financial information; an external product catalog in an e-commerce domain for a structured schema concerning purchasing and selling products; etc.). In some implementations, a generative AI model generates 504 the appropriate spoken text when in-context examples are provided in the prompt during generation. Examples collected from a different, external data source (e.g., external data source 234), but still in the same domain, are applicable for generative AI model 228 to generate 504 the in-context example. Flowsheet population process 10 performs an example similarity-based search across all collected examples from external data sources and finds a predefined number of most similar examples from the external data sources. In this example, flowsheet population process 10 does not incorporate spoken text (e.g., from a given segment) in the example similarity-based search. In another example, flowsheet population process 10 does incorporate the spoken text in the example similarity-based search.

In some implementations, examples from other key/value pairs can be applied to generate in-context examples for the target key/value pair. In some implementations, the in-context example conforms to a schema different from the structured schema. For example, suppose a separate key/value pair exists for a “Safety Interventions” group with a key of “Enhanced Safety Measures” and a value of “bed alarm set”. In this example, the key/value pair includes an example of “bed checked and alarm set”. Using this example, generative AI model 228 is able to generate an in-context example concerning “bed check” to the value “bed alarm set” based on this key/value pair. In this example, despite the group difference (i.e., “Safety” vs. “Safety Interventions”), key difference (i.e., “Safety Promotion” vs. “Enhanced Safety Measures”), and value difference (i.e., “bed alarm” vs. “bed alarm set”) present between these key/value pairs, generative AI model 228 uses this external example to improves synthetic spoken text generation for the target key/value pair.

In some implementations, flowsheet population process 10 processes 510 a transcript of information to be recorded in the structured document. As discussed above, flowsheet population process 10 processes 510 an existing transcript or generates a transcript using an ASR system. In some implementations, flowsheet population process 10 selects 512, from a plurality of rows in the structured document and based on the transcript, a subset of rows relevant to the transcript of the information. Referring again to FIG. 2, flowsheet 200 is shown with various rows (e.g., rows 204). As discussed above, flowsheet population process 10 selects 512 a plurality of rows using retrieval augmented generation (RAG) with schema row selection section 104 and database 108 that stores a full schema of the flowsheet, which can include up to tens of thousands of rows of the flowsheet. In some implementations, flowsheet population process 10 uses a schema similarity-based search (e.g., schema similarity-based search section 110), to identify the most relevant subset of rows in the full schema database 108 and stores them in the reduced schema database 112 for use in connection with matching information in each segment with the appropriate row (key/value pair) in the flowsheet.

In some implementations, flowsheet population process 10 segments 514, from the transcript, a first instance of information corresponding to a key and an associated value for the first instance of information. For example and as discussed above, flowsheet population process 10 segments 514 (e.g., as shown in FIG. 1 as segmentation step 122) the transcript into contextually coherent, continuous and non-overlapping segments such as phenomes, words, or phrases, via a generative artificial intelligence (AI) model. In some implementations, segmenting 514 includes identifying pauses, changes in pitch, or other acoustic features that indicate the boundaries between different linguistic units. Effective segmentation improves the accuracy of speech recognition by ensuring that the system can better handle variations in speech, such as different speaking speeds, accents, and sentence structures. It also aids in improving the alignment between spoken words and their corresponding text, facilitating more precise transcriptions in ASR systems. To reduce variables, segmentation is kept constant for all implementations, although this is not required.

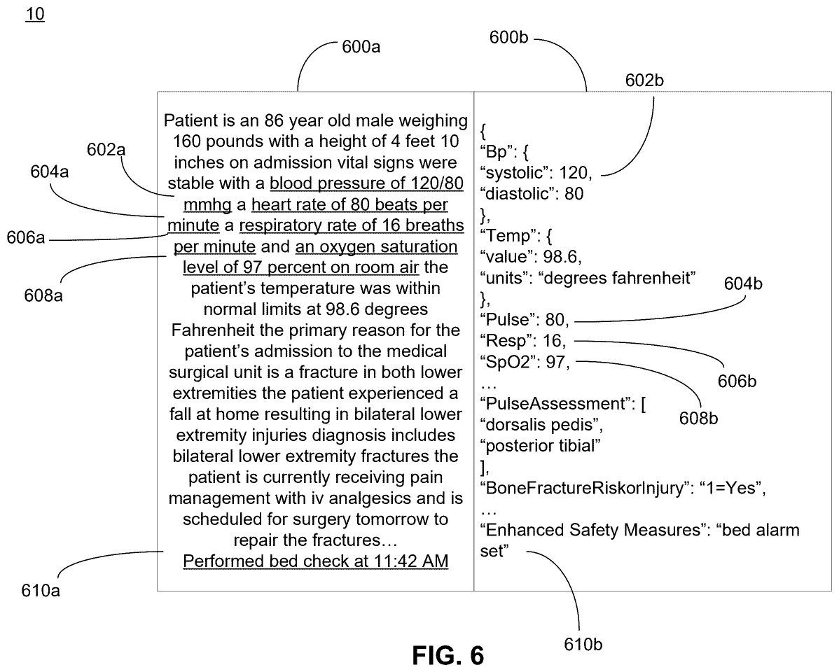

In some implementations, flowsheet population process 10 populates 516 a row in the subset of rows corresponding to the key of the first instance of information with the associated value. Referring also to FIG. 6, flowsheet population process 10 populates instances of information in a transcript into rows (key/value) of a structured document (e.g., flowsheet). A portion 600a of a transcript is shown, along with the corresponding portion 600b of the structured flowsheet. Indicated at 602a is a segment of the transcript 600a indicating a “blood pressure of 120/80 mmhg.” After processing of transcript 600a as described above, the information in this segment 602a is inserted into the key and value fields in the associated row 602b of the flowsheet 600b. In the associated row 602b in the flowsheet 600b, the key “Bp” (for blood pressure) is inserted and the values 120 (systolic) and 80 (diastolic) are entered. Likewise, the instance of information in segment 604a is inserted into the key and value fields in the associated row 604b of the flowsheet 600b. In the associated row 604b in the flowsheet 600b, the key “Pulse” and the value “80” are entered. The instance of information in segment 606a is inserted into the key and value fields in the associated row 606b of the flowsheet 600b. In the associated row 606b in the flowsheet 600b, the key “Resp” and the value “16” are entered. In this example, the instance of information in segment 608a is inserted into the key and value fields in the associated row 608b of the flowsheet 600b. In the associated row 608b in the flowsheet 600b, the key “SpO2” and the value “97” are entered. Additionally, with the newly generated in-context example for a bed alarm, the instance of information in segment 610a (i.e., “Performed bed check at 11:42 AM”) is inserted into the key “Enhanced Safety Measures” and the value “bed alarm set” is entered. In this manner, flowsheet population process 10 automatically populates rows of the flowsheet by mapping segments of a transcript to key/value portions of the schema of the flowsheet with in-context examples generated from the target key/value pair (i.e., “Enhanced Safety Measures”/“bed alarm set”) using the generative AI model (e.g., generative AI model 228).

While embodiments of the disclosure have been described mainly with regard to the application to hospital flowsheets for use by medical personnel, the system and method described herein may be utilized in connection with any environment in which freeform speech information is to be entered into a structured document.

### System Overview

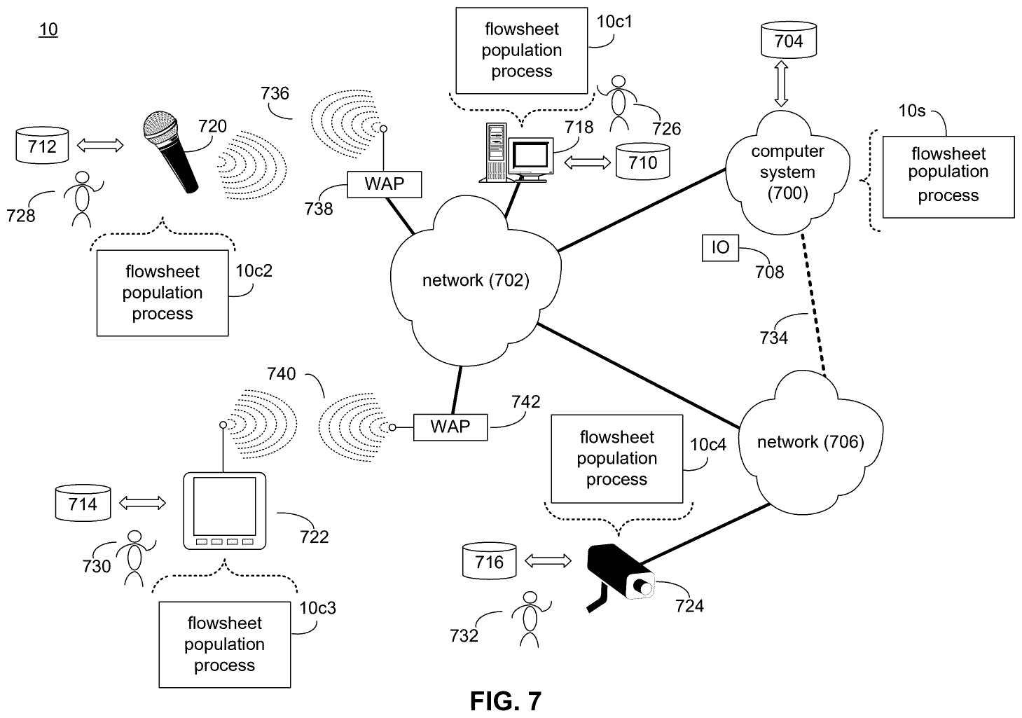

Referring to FIG. 7, there is shown a flowsheet population process 10. Flowsheet population process 10 may be implemented as a server-side process, a client-side process, or a hybrid server-side/client-side process. For example, flowsheet population process 10 may be implemented as a purely server-side process via flowsheet population process 10s. Alternatively, flowsheet population process 10 may be implemented as a purely client-side process via one or more of flowsheet population process 10c1, flowsheet population process 10c2, flowsheet population process 10c3, and flowsheet population process 10c4. Alternatively still, flowsheet population process 1044 may be implemented as a hybrid server-side/client-side process via flowsheet population process 10s in combination with one or more of flowsheet population process 10c1, flowsheet population process 10c2, flowsheet population process 10c3, and flowsheet population process 10c4.

Accordingly, flowsheet population process 10 as used in this disclosure may include any combination of flowsheet population process 10, flowsheet population process 10c1, schema matching process, flowsheet population process 10c3, and flowsheet population process 10c4.

[symbol] flowsheet population process 10s may be a server application and may reside on and may be executed by a computer system 700, which may be connected to network 702 (e.g., the Internet or a local area network). Computer system 700 may include various components, examples of which may include but are not limited to: a personal computer, a server computer, a series of server computers, a mini computer, a mainframe computer, one or more Network Attached Storage (NAS) systems, one or more Storage Area Network (SAN) systems, one or more Platform as a Service (PaaS) systems, one or more Infrastructure as a Service (IaaS) systems, one or more Software as a Service (SaaS) systems, a cloud-based computational system, and a cloud-based storage platform.

A SAN includes one or more of a personal computer, a server computer, a series of server computers, a minicomputer, a mainframe computer, a RAID device and a NAS system. The various components of computer system 700 may execute one or more operating systems.

The instruction sets and subroutines of computational cost reduction process 10s, which may be stored on storage device 704 coupled to computer system 700, may be executed by one or more processors (not shown) and one or more memory architectures (not shown) included within computer system 700. Examples of storage device 704 may include but are not limited to: a hard disk drive; a RAID device; a random-access memory (RAM); a read-only memory (ROM); and all forms of flash memory storage devices.

Network 702 may be connected to one or more secondary networks (e.g., network 704), examples of which may include but are not limited to: a local area network; a wide area network; or an intranet, for example.

Various IO requests (e.g., IO request 708) may be sent from flowsheet population process 10s, flowsheet population process 10c1, flowsheet population process 10c2, flowsheet population process 10c3 and/or flowsheet population process 10c4 to computer system 700. Examples of IO request 708 may include but are not limited to data write requests (i.e., a request that content be written to computer system 700) and data read requests (i.e., a request that content be read from computer system 700).

The instruction sets and subroutines of flowsheet population process 10c1, flowsheet population process 10c2, flowsheet population process 10c3 and/or flowsheet population process 10c4, which may be stored on storage devices 710, 712, 714, 716 (respectively) coupled to client electronic devices 718, 720, 722, 724 (respectively), may be executed by one or more processors (not shown) and one or more memory architectures (not shown) incorporated into client electronic devices 718, 720, 722, 724 (respectively). Storage devices 710, 712, 714, 716 may include but are not limited to: hard disk drives; optical drives; RAID devices; random access memories (RAM); read-only memories (ROM), and all forms of flash memory storage devices. Examples of client electronic devices 718, 720, 722, 724 may include, but are not limited to, personal computing device 718 (e.g., a smart phone, a personal digital assistant, a laptop computer, a notebook computer, and a desktop computer), audio input device 720 (e.g., a handheld microphone, a lapel microphone, an embedded microphone (such as those embedded within eyeglasses, smart phones, tablet computers and/or watches) and an audio recording device), display device 722 (e.g., a tablet computer, a computer monitor, and a smart television), a hybrid device (e.g., a single device that includes the functionality of one or more of the above-references devices; not shown), an audio rendering device (e.g., a speaker system, a headphone system, or an earbud system; not shown), and a dedicated network device (not shown).

Users 726, 728, 730, 732 may access computer system 700 directly through network 702 or through secondary network 706. Further, computer system 700 may be connected to network 702 through secondary network 706, as illustrated with link line 734.

The various client electronic devices (e.g., client electronic devices 718, 720, 722, 724) may be directly or indirectly coupled to network 702 (or network 706). For example, personal computing device 718 is shown directly coupled to network 702 via a hardwired network connection. Further, machine vision input device 724 is shown directly coupled to network 706 via a hardwired network connection. Audio input device 722 is shown wirelessly coupled to network 702 via wireless communication channel 736 established between audio input device 720 and wireless access point (i.e., WAP) 738, which is shown directly coupled to network 702. WAP 738 may be, for example, an IEEE 802.11a, 802.11b, 802.11g, 802.11n, Wi-Fi, and/or any device that is capable of establishing wireless communication channel 736 between audio input device 720 and WAP 738. Display device 722 is shown wirelessly coupled to network 702 via wireless communication channel 740 established between display device 722 and WAP 742, which is shown directly coupled to network 702.

The various client electronic devices (e.g., client electronic devices 718, 720, 722, 724) may each execute an operating system, wherein the combination of the various client electronic devices (e.g., client electronic devices 718, 720, 722, 724) and computer system 700 may form a modular system.

### General

As will be appreciated by one skilled in the art, the present disclosure may be embodied as a method, a system, or a computer program product. Accordingly, the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present disclosure may take the form of a computer program product on a computer-usable storage medium having computer-usable program code embodied in the medium.

Any suitable computer usable or computer readable medium may be used. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. The computer-usable or computer-readable medium may also be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, RF, etc.

Computer program code for carrying out operations of the present disclosure may be written in an object-oriented programming language. However, the computer program code for carrying out operations of the present disclosure may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network/a wide area network/the Internet.

The present disclosure is described with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, may be implemented by computer program instructions. These computer program instructions may be provided to a processor of a general-purpose computer/special purpose computer/other programmable data processing apparatus, such that the instructions, which execute via the processor of the computer or other programmable data processing apparatus, create means for implementing the functions/acts specified in the flowchart and/or block diagram block or blocks.

These computer program instructions may also be stored in a computer-readable memory that may direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams in the figures may illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, not at all, or in any combination with any other flowcharts depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. As used herein, the language “at least one of A and B” (and the like) as well as “at least one of A or B” (and the like) should be interpreted as covering only A, only B, or both A and B, unless the context clearly indicates otherwise. The language “one or more of A and B” (and the like) as well as “one or more of A or B” (and the like) should be interpreted as covering only A, only B, or both A and B, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. A computer-implemented method, executed on a computing device, comprising:
processing a structured document conforming to a schema and having a plurality of rows, each row comprising a key and a value;
identifying a target key/value pair within the structured document for a target instance of information; and
generating an in-context example of spoken text representative of the target instance of information corresponding to the target key/value pair using a generative artificial intelligence (AI) model.

2. The computer-implemented method of claim 1, wherein the in-context example conforms to a schema different from the structured schema.

3. The computer-implemented method of claim 1, wherein generating the in-context example includes processing a plurality of examples from an external data source by determining a similarity between the plurality of examples and the target key/value pair and generating a prompt for the generative AI model that includes a predefined number of most similar examples from the plurality of examples.

4. The computer-implemented method of claim 1, wherein generating the in-context example includes converting the target key/value pair into the in-context example of spoken text using the generative AI model.

5. The computer-implemented method of claim 1, further comprising:
processing a transcript of information to be recorded in the structured document.

6. The computer-implemented method of claim 5, further comprising:
selecting, from a plurality of rows in the structured document and based on the transcript, a subset of rows relevant to the transcript of the information.

7. The computer-implemented method of claim 6, further comprising:
segmenting, from the transcript, a first instance of information corresponding to a key and an associated value for the first instance of information.

8. The computer-implemented method of claim 7, further comprising:
populating a row in the subset of rows corresponding to the key of the first instance of information with the associated value.

9. The computer-implemented method of claim 1, wherein the structured document is a flowsheet associated with patient monitoring within a healthcare facility.

10. The computer-implemented method of claim 9, wherein each key of the flowsheet corresponds to an instance of patient information.

11. A computing system comprising:
a memory; and
a processor to:
process a structured document conforming to a schema and having a plurality of rows, each row comprising a key and a value;
identify a target key/value pair within the structured document for a target instance of information; and
generate an in-context example of spoken text representative of the target instance of information corresponding to the target key/value pair using a generative artificial intelligence (AI) model, wherein generating the in-context example includes converting the target key/value pair into the in-context example of spoken text using the generative AI model.

12. The computing system of claim 11, wherein the in-context example conforms to a schema different from the structured schema.

13. The computing system of claim 11, wherein generating the in-context example includes processing a plurality of examples from an external data source by determining a similarity between the plurality of examples and the target key/value pair and generating a prompt for the generative AI model that includes a predefined number of most similar examples from the plurality of examples.

14. A computer program product residing on a non-transitory computer readable medium having a plurality of instructions stored thereon which, when executed by a processor, cause the processor to perform operations comprising:
processing a structured document conforming to a schema and having a plurality of rows, each row comprising a key and a value;
identifying a target key/value pair within the structured document for a target instance of information; and
generating an in-context example of spoken text representative of the target instance of information corresponding to the target key/value pair using a generative artificial intelligence (AI) model, wherein the in-context example is generated from a data source external from the structured document conforming to the schema.

15. The computer program product of claim 14, wherein generating the in-context example includes processing a plurality of examples from an external data source by determining a similarity between the plurality of examples and the target key/value pair and generating a prompt for the generative AI model that includes a predefined number of most similar examples from the plurality of examples.

16. The computer program product of claim 14, wherein generating the in-context example includes converting the target key/value pair into the in-context example of spoken text using the generative AI model.

17. The computer program product of claim 14, wherein the operations further comprise:
processing a transcript of information to be recorded in the structured document.

18. The computer program product of claim 17, wherein the operations further comprise:
selecting, from a plurality of rows in the structured document and based on the transcript, a subset of rows relevant to the transcript of the information.

19. The computer program product of claim 18, wherein the operations further comprise:
populating a row in the subset of rows corresponding to the key of the first instance of information with the associated value.

20. The computer program product of claim 14, wherein each key of the flowsheet corresponds to an instance of patient information.
